(19) United States
(12) Patent Application Publication
(10) Pub. No.: US 2010/0087858 A1
(43) Pub. Date: Apr. 8, 2010

(54) DYNAMIC CONNECTOR FOR SPINAL STABILIZATION AND METHOD OF USE

(76) Inventor: M. Samy Abdou, San Diego, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/562,867

(22) Filed: Sep. 18, 2009

Related U.S. Application Data
(60) Provisional application No. 61/192,706, filed on Sep. 18, 2008.
(60) Provisional application No. 61/212,408, filed on Apr. 10, 2009.

(51) Int. Cl. A61B 17/70

(52) U.S. Cl. 606/246

(57) ABSTRACT

Disclosed are novel dynamic rod systems and methods that provide robust fixation and a reliable resistor to motion. A first end of a movable orthopedic implant is attached onto a first vertebral bone of a functional spinal unit and a second end onto a second vertebral bone of a functional spinal unit. The implant has a longitudinal axis and permits movement of the attached vertebral bones along that longitudinal axis. The device may contain a member that is adapted to resist movement wherein longitudinal movement of a first device member relative to a second device member produces rotational movement about the long axis the device within the resisting member.

### REFERENCE TO PRIORITY DOCUMENTS

This application claims priority of co-pending U.S. Provisional Patent Application Ser. No. 61/192,706, filed Sep. 18, 2008, and co-pending U.S. Provisional Patent Application Ser. No. 61/212,408, filed Apr. 10, 2009. Priority of the aforementioned filing dates is hereby claimed. The disclosure of each Provisional patent Application is hereby incorporated by reference in its entirety.

### BACKGROUND

Progressive constriction of the central canal within the spinal column is a predictable consequence of aging. As the spinal canal narrows, the nerve elements that reside within it become progressively more crowded. Eventually, the canal dimensions become sufficiently small so as to significantly compress the nerve elements and produce pain, weakness, sensory changes, clumsiness and other manifestation of nervous system dysfunction.

Constriction of the canal within the lumbar spine is termed lumbar stenosis. This condition is common in the elderly and causes a significant proportion of the low back pain, lower extremity pain, lower extremity weakness, limitation of mobility and the high disability rates that afflict this age group. With aging and spinal degeneration, displacement of the vertebral bones in the horizontal may occur and the condition is termed Sponylolisthesis. Spondylolisthesis exacerbates the extent of nerve compression within the spinal canal since misalignment of the vertebral bones will further reduce the size of the spinal canal.

Relief for the compressed nerves can be achieved by the surgical removal of the bone and ligamentous structures that constrict the spinal canal. However, decompression of the spinal canal can further weaken the facet joints and increase the possibility of additional aberrant vertebral movement in the horizontal plane. Thus, decompression can worsen the extent of spondylolisthesis or produce spondylolisthesis in an otherwise normally aligned functional spinal unit (FSU). After decompression, surgeons will commonly fuse and immobilize the adjacent spinal bones in order to prevent the development of post-operative vertebral misalignment and spondylolisthesis.

Since fusion will often place additional load on the adjacent spinal segments and hasten degeneration of those levels, it is of significant clinical interest to develop an orthopedic implant that would preventing aberrant movement between adjacent vertebral bones while permitting decompression of the nerve elements without concurrent fusion.

U.S. Pat. Nos. 5,011,484, 5,092,866, 5,180,393, 5,387,213, 5,540,688, 5,562,737, 5,672,175, 5,725,582, 5,961,516, 5,984,923, 6,296,643, 6,248,106, 6,645,207, 6,682,530, 6,966,910, 2004/0236329, 2005/0171543, 2005/0177156 and others have disclosed numerous devices for the dynamic stabilization of the spine. As shown in these patents, current dynamic stabilization devices are large, multi-segmental spring-based devices that are placed using traditional surgical approaches to the vertebral column. Further, clinical experience with these devices has produced mechanical failure because of rod breakage in multiple systems.

### SUMMARY

Disclosed are novel dynamic rod systems and methods that provide robust fixation and a reliable resistor to motion. A first end of a movable orthopedic implant is attached onto a first vertebral bone of a functional spinal unit and a second end onto a second vertebral bone of a functional spinal unit. The implant has a longitudinal axis and permits movement of the attached vertebral bones along that longitudinal axis. The device may contain a member that is adapted to resist movement wherein longitudinal movement of a first device member relative to a second device member produces rotational movement about the long axis the device within the resisting member. In an embodiment, a spinal grove is used to convert the longitudinal movement of the device members and the rotational movement of the resistor. There is also provided resistance to longitudinal movement in either direction using a single resistor to motion, wherein the resistor is positioned outside of the attachments points of the device to the vertebral bones. This provides maximal strength of the device.

Other features and advantages should be apparent from the following description of various embodiments, which illustrate, by way of example, the principles of the disclosed devices and methods.

### BRIEF DESCRIPTION OF THE DRAWINGS

FIG. 1 shows a diagrammatic representation of a spinal vertebral bone in multiple views.

FIGS. 2A and 2B illustrate a functional spinal unit (FSU).

FIG. 3A illustrates three vertebral bones with relatively normal alignment.

FIG. 3B shows the anterior displacement of the middle bone relative to the inferior-most bone.

FIGS. 4A and 4B illustrate perspective and partial sectional views of a first device embodiment.

FIG. 5 shows the dissembled device of FIGS. 4A and 4B.

FIGS. 6A and 6B illustrate a segment and pivot member on each end of device of FIGS. 4A and 4B.

FIGS. 7A and 7B show a collar of the device.

FIGS. 8A and 8B show cross sectional views of the disassembled device.

FIG. 9 shows a perspective view of the assembled device.

FIGS. 10A and 10B show the assembled malleable pivot member and a partial section view of the pivot member.

FIGS. 11 and 12, and 13A show views of the pivot member.

FIG. 13B show an exploded view of a device.

FIGS. 14 shows the device using a curvilinear channel.

FIGS. 15A and 15B show cross-sectional views of different embodiments.

FIG. 16 illustrates a prospective view of the device attached to vertebral bone.

FIG. 17 shows a sectional view through the device.

FIG. 18 illustrates vertebral flexion with a rigid bone fastener used to attach the device to vertebra V2 and a movable bone fastener used to attach the device to vertebra V1.

### DETAILED DESCRIPTION

In order to promote an understanding of the principals of the invention, reference is made to the drawings and the embodiments illustrated therein. Nevertheless, it will be understood that the drawings are illustrative and no limitation of the scope of the invention is thereby intended. Any such alterations and further modifications in the illustrated embodiments, and any such further applications of the principles of the invention as illustrated herein are contemplated as would normally occur to one of ordinary skill in the art.

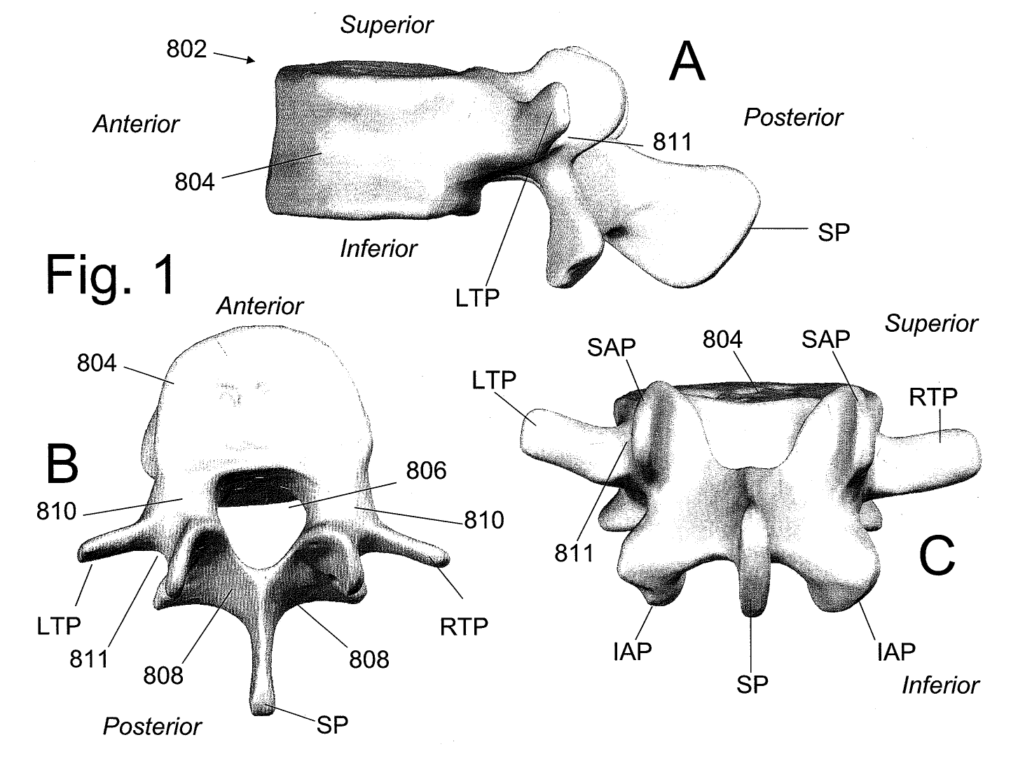

FIG. 1 shows a diagrammatic representation of a spinal vertebral bone 802 in multiple views. For clarity of illustration, the vertebral bone of FIG. 1 and those of other illustrations presented in this application are represented schematically and those skilled in the art will appreciate that actual vertebral bodies may include anatomical details that are not shown in these figures. Further, it is understood that the vertebral bones at a given level of the spinal column of a human or animal subject will contain anatomical features that may not be present at other levels of the same spinal column. The illustrated vertebral bones are intended to generically represent vertebral bones at any spinal level without limitation. Thus, the disclosed devices and methods may be applied at any applicable spinal level.

Vertebral bone 802 contains an anteriorly-placed vertebral body 804, a centrally placed spinal canal and 806 and posteriorly-placed lamina 808. The pedicle (810) segments of vertebral bone 802 form the lateral aspect of the spinal canal and connect the laminas 808 to the vertebral body 804. The spinal canal contains neural structures such as the spinal cord and/or nerves. A midline protrusion termed the spinous process (SP) extends posteriorly from the medial aspect of laminas 808. A protrusion extends laterally from each side of the posterior aspect of the vertebral bone and is termed the transverse process (TP). A right transverse process (RTP) extends to the right and a left transverse process (LTP) extends to the left. A superior protrusion extends superiorly above the lamina on each side of the vertebral midline and is termed the superior articulating process (SAP). An inferior protrusion extends inferiorly below the lamina on each side of the vertebral midline and is termed the inferior articulating process (IAP). Note that the posterior aspect of the pedicle can be accessed at an indentation 811 in the vertebral bone between the lateral aspect of the SAP and the medial aspect of the transverse process (TP). In surgery, it is common practice to anchor a bone fastener into the pedicle portion of a vertebral bone by inserting the fastener through indentation 811 and into the underlying pedicle.

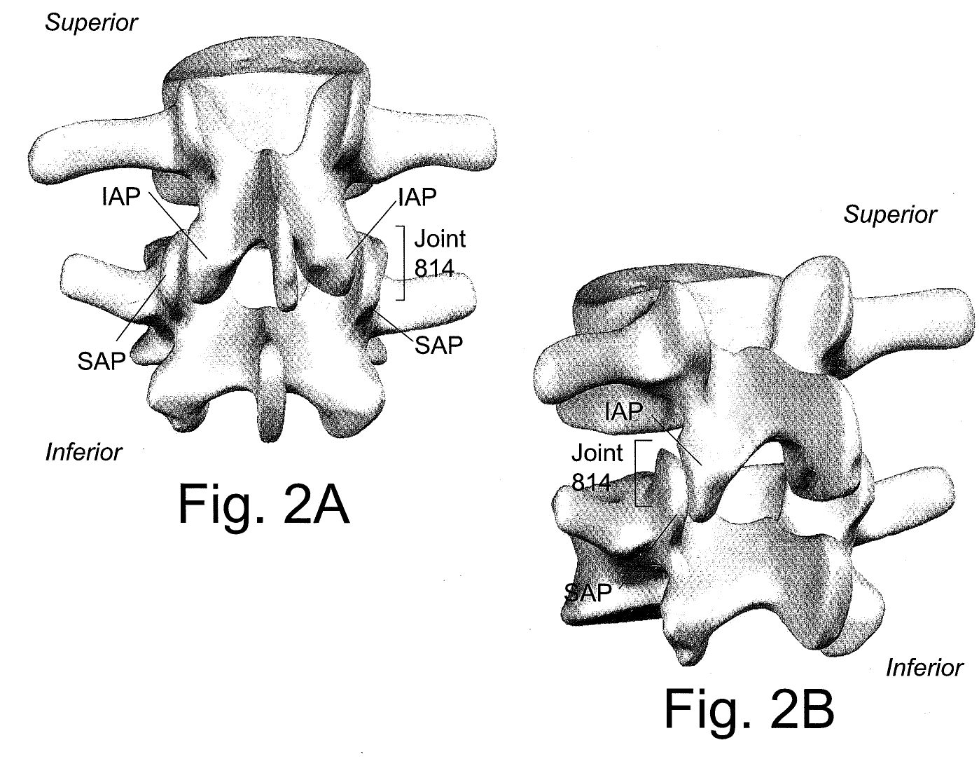

FIGS. 2A and 2B illustrate a functional spinal unit (FSU), which includes two adjacent vertebrae and the intervertebral disc between them. The intervertebral disc resides between the inferior surface of the upper vertebral body and the superior surface of the lower vertebral body. (Note that a space is shown in FIG. 2 where intervertebral disc would reside.) FIG. 2A shows the posterior surface of the adjacent vertebrae and the articulations between them while FIG. 2B shows an oblique view. Note that FSU contains a three joint complex between the two vertebral bones, with the intervertebral disc comprising the anterior joint. The posterior joints include a facet joint 814 on each side of the midline, wherein the facet joint contains the articulation between the IAP of the superior vertebral bone and the SAP of the inferior bone.

The preceding illustrations and definitions of anatomical structures are known to those of ordinary skill in the art. They are illustrated in more detail in Atlas of Human Anatomy, by Frank Netter, third edition, Icon Learning Systems, Teterboro, N.J. The text is hereby incorporated by reference in its entirety.

In the functional spinal unit, a substantial portion (up to 80%) of the vertical load is borne by the intervertebral disc and the anterior column. (The term “vertical load” refers to the load transmitted in the vertical plane through the erect human spine. The “anterior column” is used here to designate that portion of the vertebral body and/or FSU that is situated anterior to the posterior longitudinal ligament and includes the posterior longitudinal ligament. Thus, its use in this application encompasses both the anterior and middle column of Denis. See The three column spine and its significance in the classification of acute thoracolumbar spinal injuries. By Denis, F. Spine 1983 November-December; 8(8):817-31. The article is incorporated by reference in its entirety.) Conversely, a substantial portion of load transmitted through the functional spine unit in the horizontal plane is borne by the facet joint and the posterior column. (The “posterior column” is used here to designate that portion of the vertebral body and/or FSU that is situated posterior to the posterior longitudinal ligament.)

Generally, the forces acting in the horizontal plane are aligned to cause an anterior displacement of the superior vertebral body relative to the inferior vertebral body of a functional spinal unit. These forces are counteracted by the facet joints which are formed by the abutment surfaces of the IAP of the superior vertebral bone and the SAP of the inferior bone.

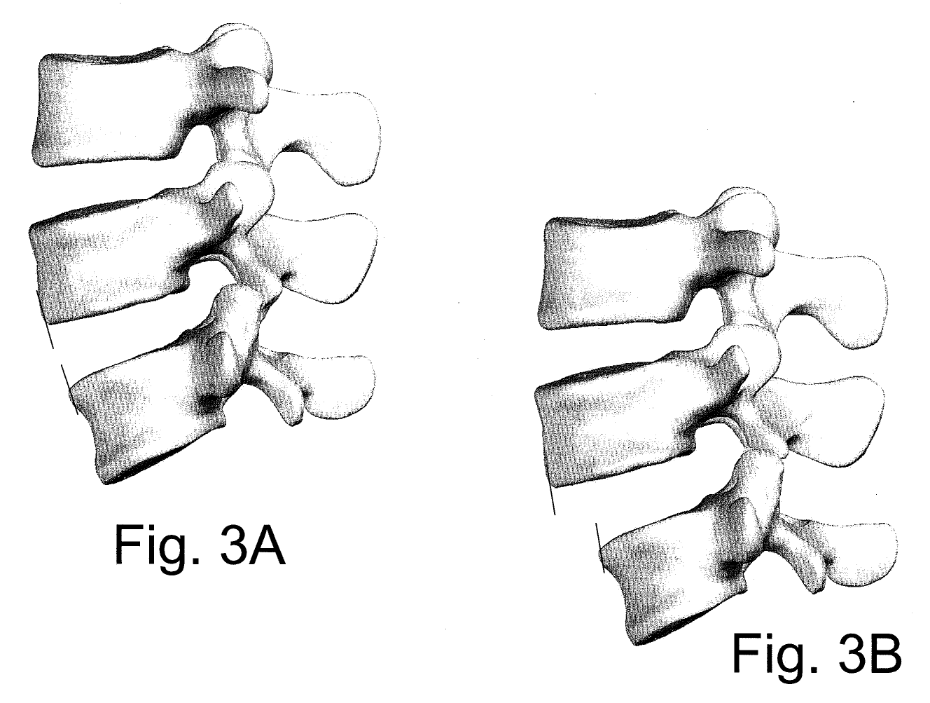

In a healthy spine functioning within physiological parameters, the two facet joints of an FSU collectively function to prevent aberrant relative movement of the vertebral bones in the horizontal plane. With aging and spinal degeneration, displacement of the vertebral bones in the horizontal may occur and the condition is termed Sponylolisthesis. FIG. 3A illustrates three vertebral bones with relatively normal alignment, whereas FIG. 3B shows the anterior displacement of the middle bone relative to the inferior-most bone. In the illustration, the vertebral column of FIG. 3B is said to have an anterior spondylolisthesis of the middle vertebral bone relative to the inferior-most vertebral bone, since the middle bone is anteriorly displaced relative to the inferior bone.

A spondylolisthesis can be anterior, as shown in FIG. 3B, or posterior wherein a superior vertebral bone of a functional spinal unit is posteriorly displaced in the horizontal plane relative to the inferior vertebral bone. Anterior Sponylolisthesis is more common and more clinically relevant than posterior Sponylolisthesis. (Sponylolisthesis can be further classified based on the extent of vertebral displacement. See Principles and practice of spine surgery by Vaccaro, Bets, Zeidman; Mosby press, Philadelphia, Pa.; 2003. The text is incorporated by reference in its entirety.)

With degeneration of the spine, constriction of the spinal canal (spinal stenosis) and impingement of the contained nerve elements frequently occurs and is termed spinal stenosis. Spondylolisthesis exacerbates the extent of nerve compression within the spinal canal since misalignment of bone within the horizontal plane will further reduce the size of the spinal canal. Relief for the compressed nerves can be achieved by the surgical removal of the bone and ligamentous structures that constrict the spinal canal. However, decompression of the spinal canal can further weaken the facet joints and increase the possibility of additional aberrant vertebral movement in the horizontal plane and worsen the extent of spondylolisthesis or produce spondylolisthesis in an otherwise normally aligned FSU. After decompression, surgeons will commonly fuse and immobilize the adjacent spinal bones in order to prevent the development of post-operative vertebral misalignment and spondylolisthesis.

It is a goal of the present disclosure to attach a first end of a movable orthopedic implant device onto a first vertebral bone of a functional spinal unit and a second end onto a second vertebral bone of a functional spinal unit. The device has a longitudinal axis and permits movement of the attached vertebral bones along that longitudinal axis. The device may contain a member that is adapted to resist movement wherein longitudinal movement of a first device member relative to a second device member produces rotational movement about the long axis the device within the resisting member. In an embodiment, a spinal grove is used to convert the longitudinal movement of the device members and the rotational movement of the resistor. It is an additional goal of the current invention to provide resistance to longitudinal movement in either direction using a single resistor to motion, wherein the resistor is positioned outside of the attachments points of the device to the vertebral bones. This would provide maximal strength of the device.

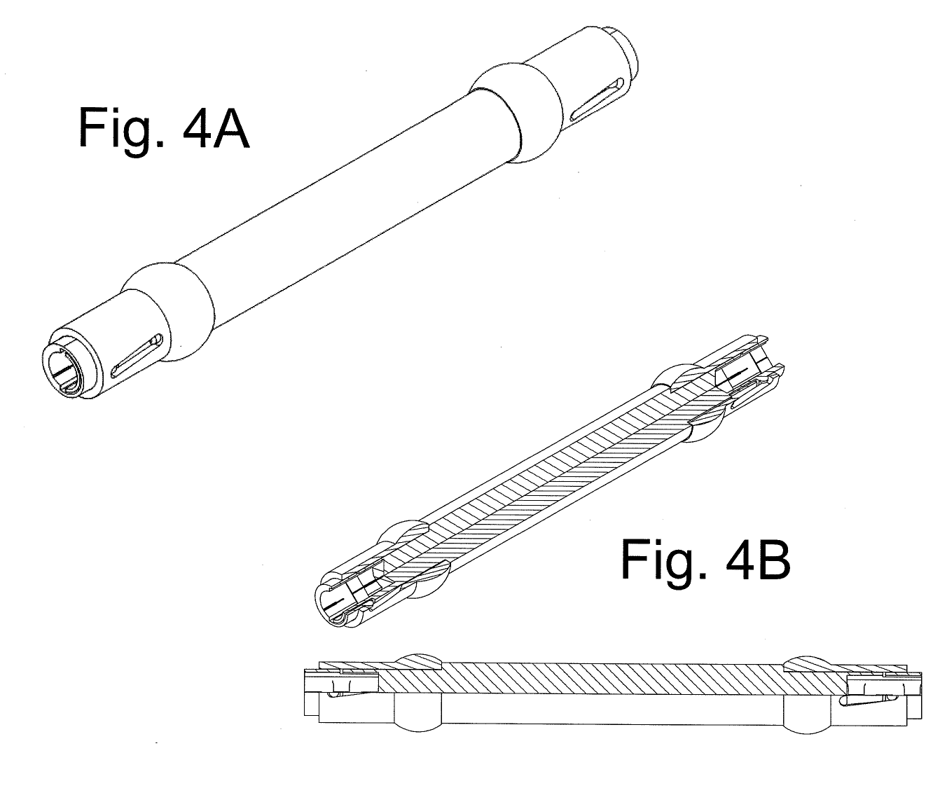

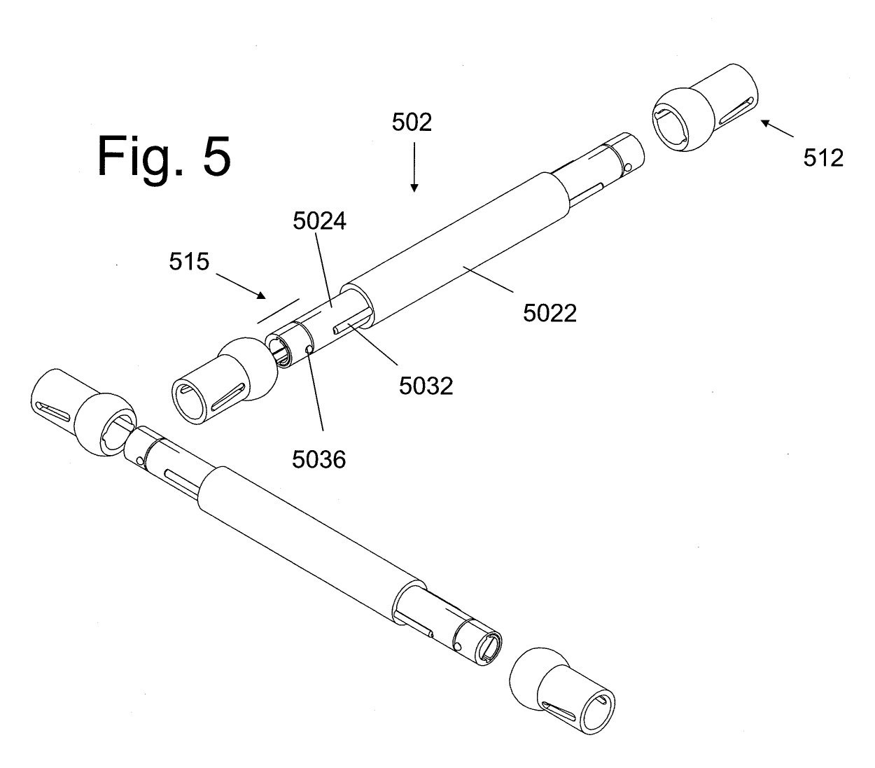

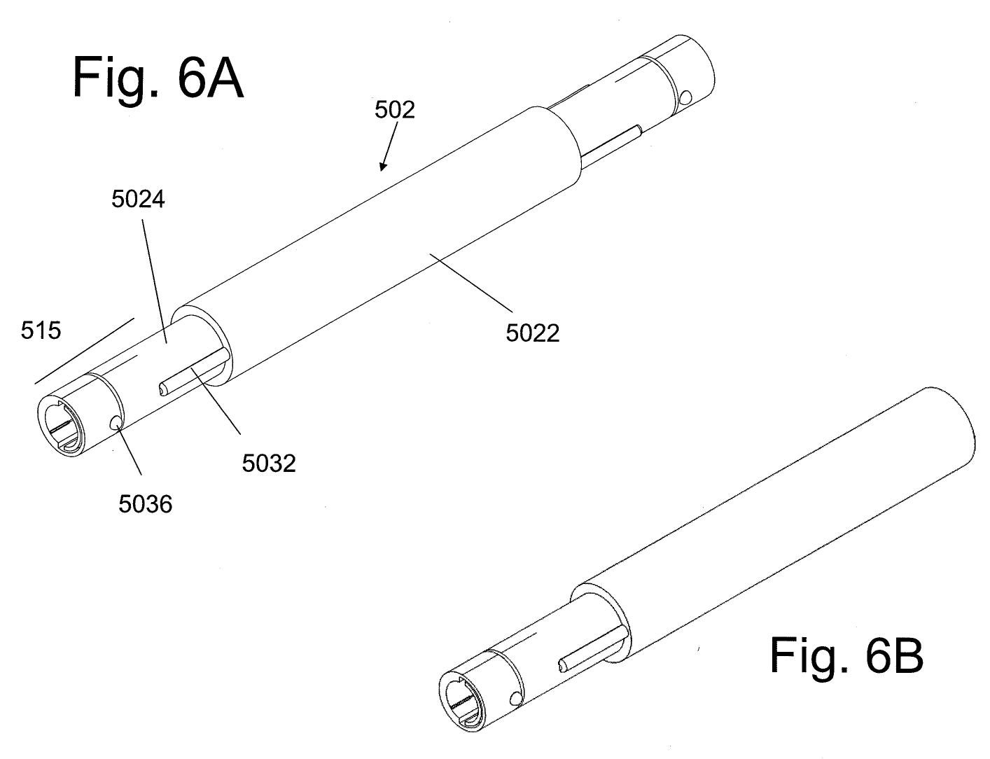

FIGS. 4A and 4B illustrate perspective and partial sectional views of a first device embodiment. FIG. 5 shows the dissembled device. The device is comprised of a rod member 502 and a movable collar 512. Rod member 502 is shown in FIGS. 6A and 6B and has a central segment 5022. On at least one end, member 502 has a smaller-diameter segment 5024. Distal to segment 5024 is a malleable pivot member 515. The rod further contains a cylindrical protrusion 5032 on the outer surface of the smaller-diameter solid segment 5024. It also contains a spherical protrusion 5036 on the distal end of the malleable pivot member 515. While FIG. 6A illustrates a segment 5024 and pivot member 515 on each end of device 502, it is understood that device 502 may alternatively have only one end containing segment 5024 and pivot member 515 with a solid second end—as shown in FIG. 6B.

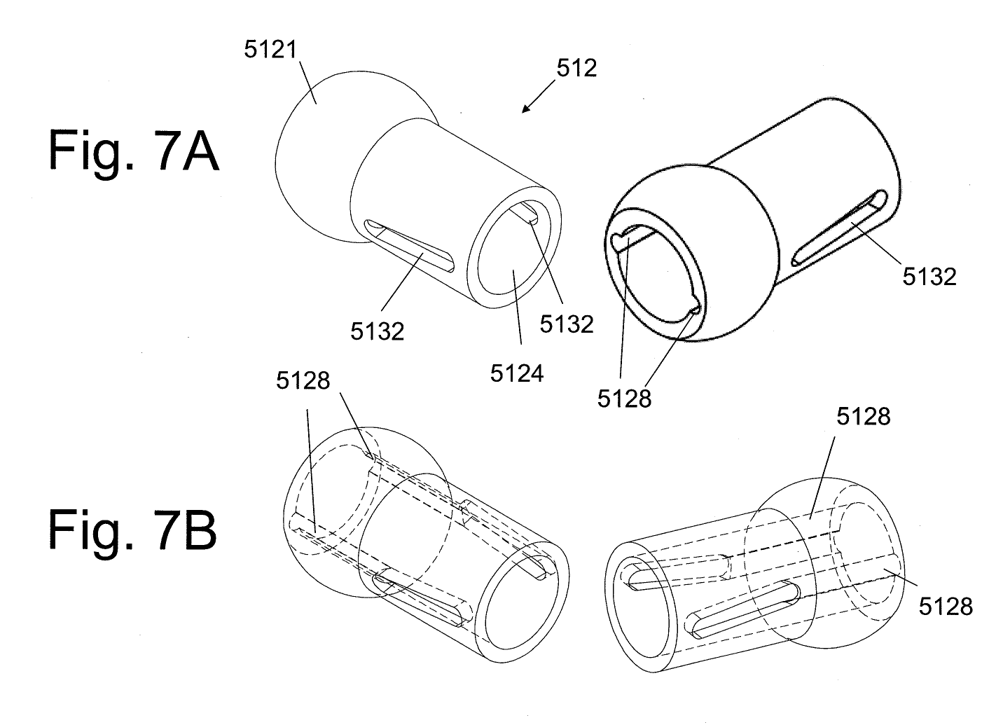

Collar 512 is shown in FIGS. 7A and 7B. The collar contains a spherical protrusion 5121, cylindrical segment 5122 and an internal bore 5124. Bore 5124 is adapted to accept an end segment of rod 502. The collar also contains at lease one full thickness channel 5132 on the cylindrical segment 5122, which preferably, but not necessarily, partially revolves around segment 5122 and forms a helical slot. Channel 5132 is adapted to contain and guide spherical protrusion 5036 of the distal end of pivot member 515 of rod 502. Collar 512 also contains a partial thickness channel 5128 on the internal aspect of the spherical protrusion 5121, wherein the channel 5128 is formed on the wall of and communicates with bore 5124. Channel 5128 is partial thickness and does not extend onto the outer spherical wall of spherical protrusion 5121. Preferably, channel 5128 is straight channel that is shaped in semi-cylindrical configuration, wherein a first end of the channel 5128 extends to the open end of spherical protrusion 5121 (FIG. 7A) and a second end of channel 5128 extends to the proximal end of full thickness channel 5132. Straight channel 5128 is adapted to contain and guide cylindrical protrusion 5032 of rod 502 and ensures that collar 512 can translate parallel to the long axis of rod 502 without rotating about the long axis of rod 502.

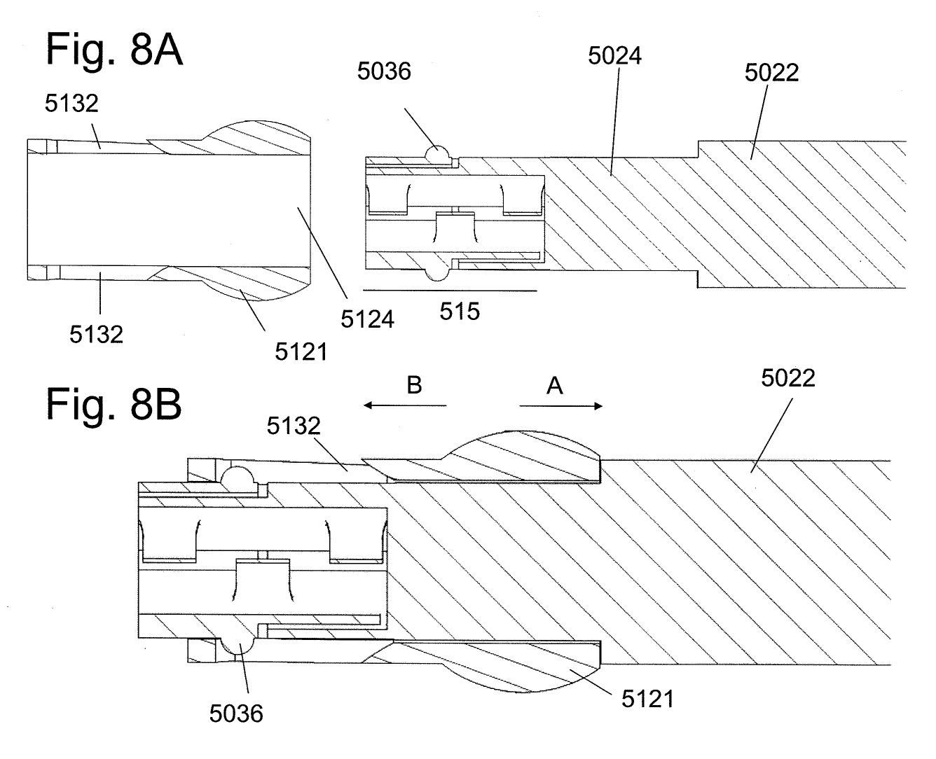

FIG. 8A shows a cross sectional view of the disassembled device with the rod 502 and collar 512 separated, while FIG. 8B shows the assembled device. Note that the section views are obtained along the direction of channel 5132. In this way, protrusion 5036 is shown in channel 5132. However, channel 5128 of collar 512 and protrusion 5032 of rod 502 are out of plane and can not be seen in the illustrated section.

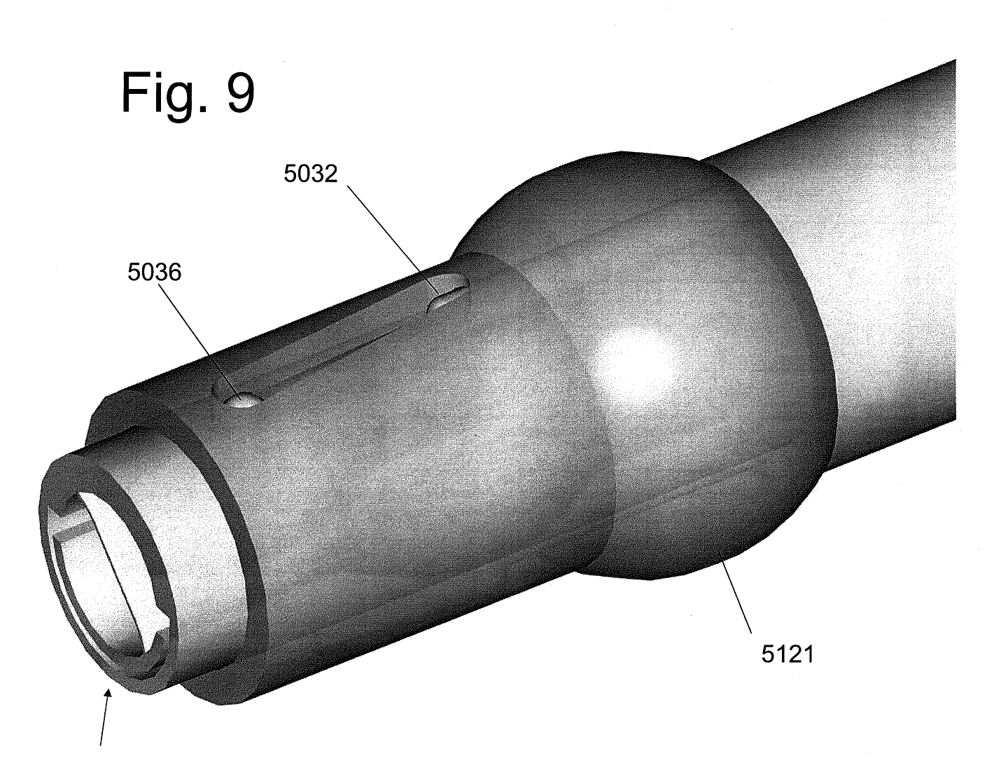

With reference to FIG. 8B, note that spherical segment 5121 rests at the level of solid segment 5024 of rod 502. Malleable pivot member 515 resists movement of collar 512 relative to rod 502. Because the region of the resistor (i.e., pivot member 515 in this device) can be expected to be weakest region and the segment most likely to fracture with the application of load perpendicular to the long axis of the rod 502, the resistor 515 is purposely placed outside the area of greatest load bearing. Further, while seated distal to the area of greatest load bearing, the resistor may be adapted to resist movement of the collar in either direction along the long axis of the rod. This is a critical design feature and will be more fully discussed below. FIG. 9 shows a perspective view of the assembled device. For illustration purpose, the collar is shown partially lucent and permits some visualization of the underlying protrusions 5036 and 5032 of rod 502.

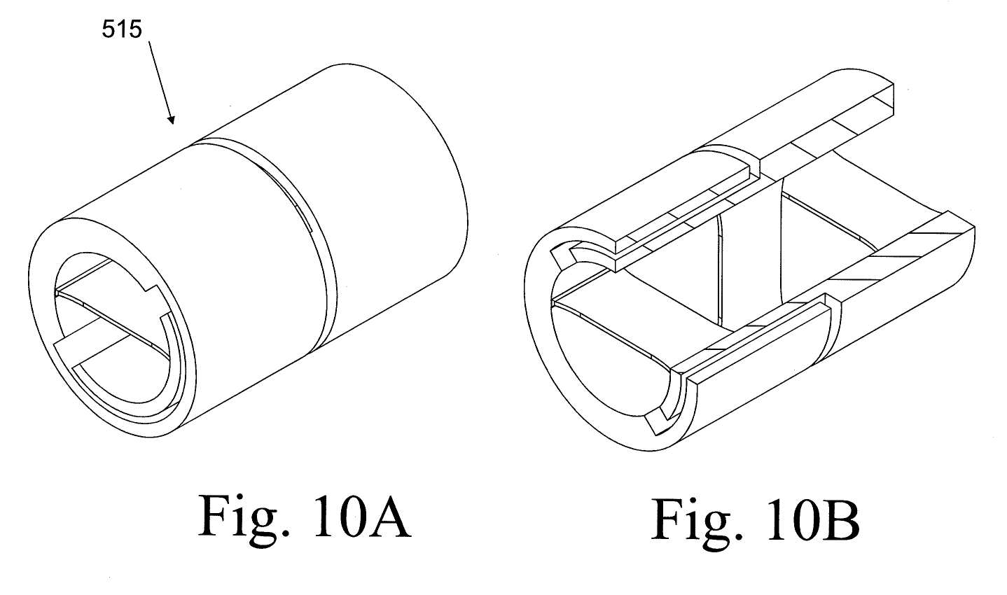

FIGS. 10A and 1013 show the assembled malleable pivot member 515 and a partial section view of member 515, respectively. The pivot member 515 is formed by a plurality of sections. Member 515 is a flexure based bearing, utilizing internal flat crossed slats 307, encapsulated in cylindrical housings 303, to provide precise rotation with low hysteresis and little frictional losses. The bearing is relatively friction-free, requires no lubrication, and is self-returning. Member 515 can resist rotational movement away from a neutral state and the extent of resistance to rotation is directly related to the extent of rotation. Member 515 has high axial stiffness.

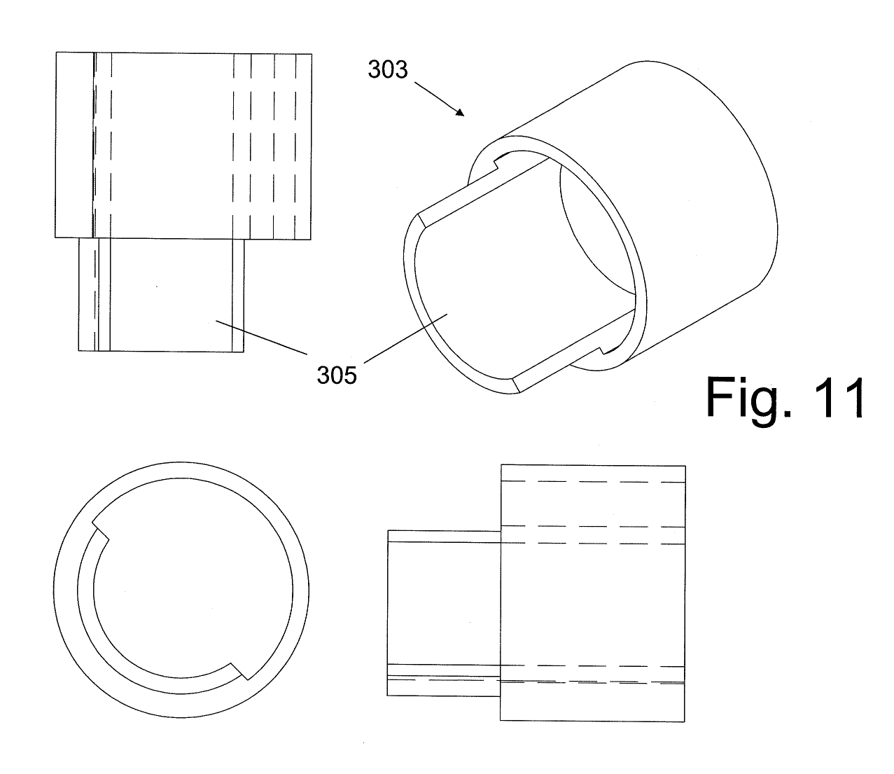

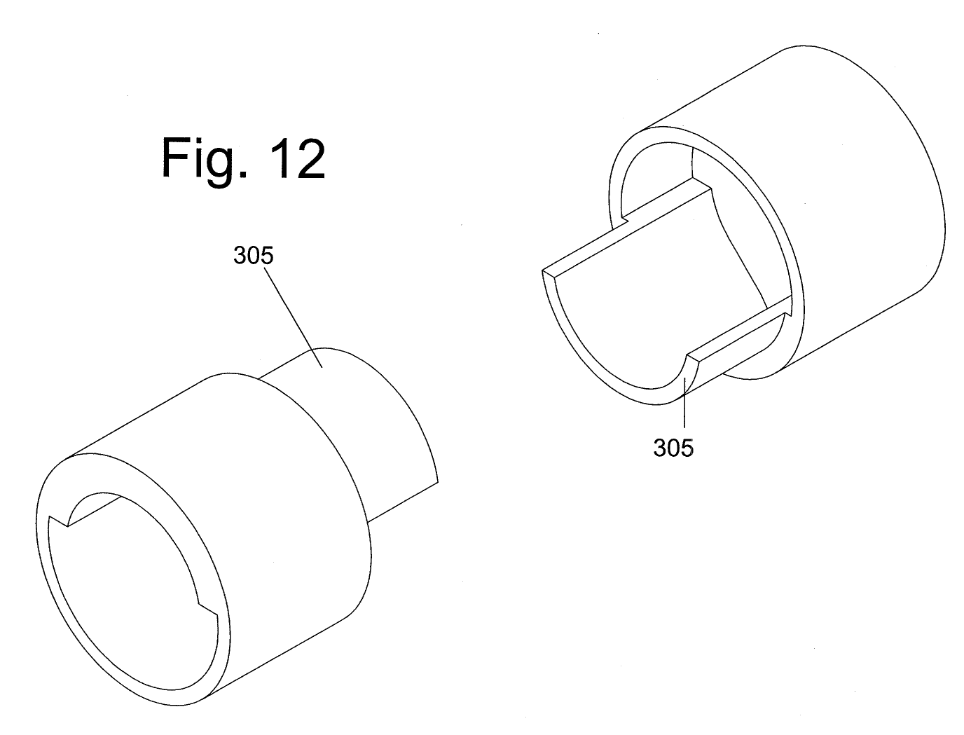

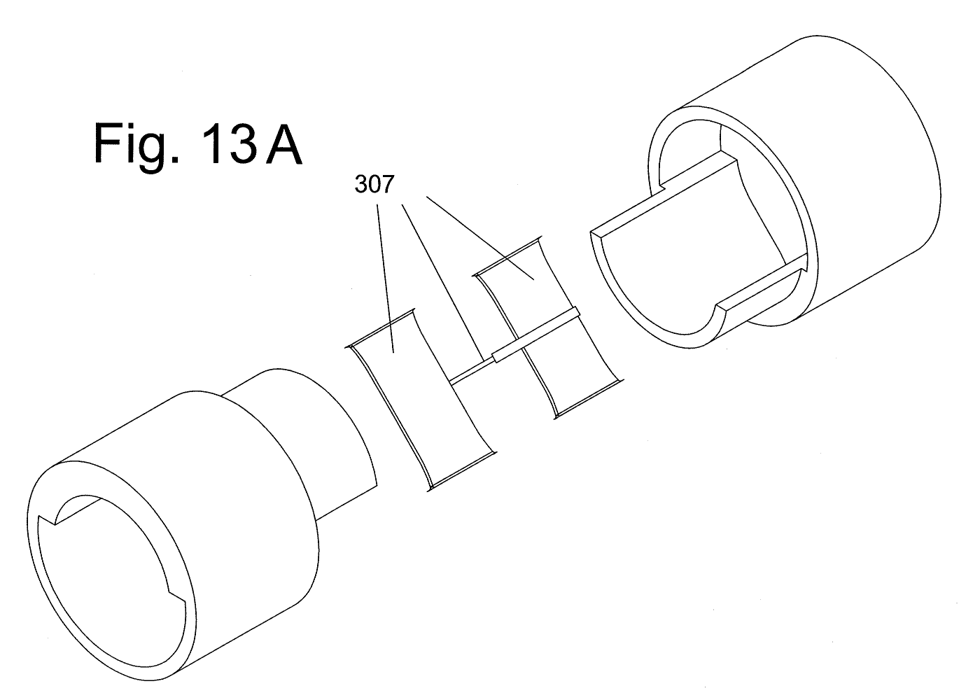

As shown in FIG. 11, the pivot member is a flexible rotational articulation that contains a first hollow cylindrical member 303 which is comprised of an outer surface with a defined radius from a longitudinal central axis, an inner surface with a defined radius from a longitudinal central axis, a defined thickness and defined internal cavity that is contained within the inner surface. At least one cylindrical tab 305 extends from one end of said member wherein the tab circumferentially extends preferably less than one hundred and eighty degrees around its longitudinal central axis.

A second hollow cylindrical member which is comprised of an outer surface with a defined radius from a longitudinal central axis, an inner surface with a defined radius from a longitudinal central axis, a defined thickness and defined internal cavity that is contained within the inner surface. At least one cylindrical tab extends from one end of said member. Preferably, but not necessarily, the first and second cylindrical members may be identical.

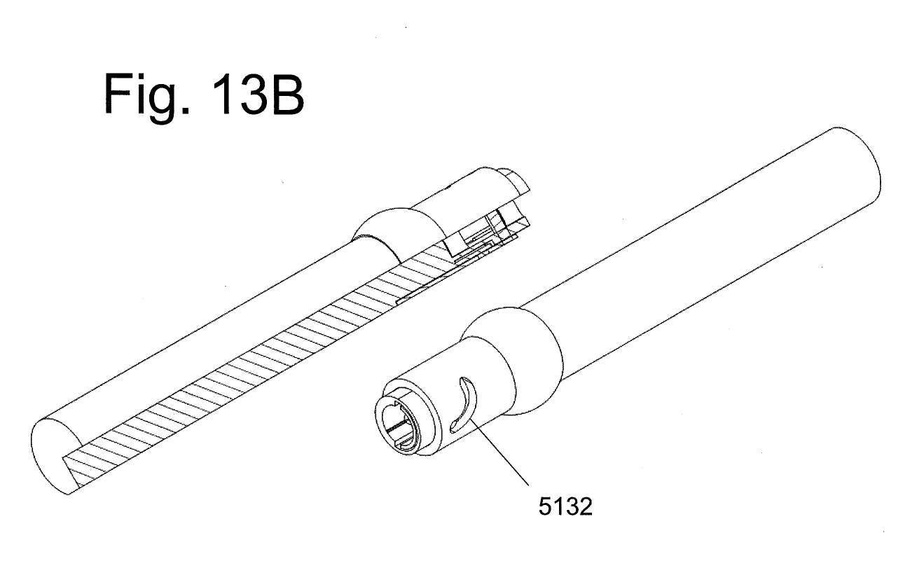

As shown in FIGS. 12 and 13A, the first and second members are axially aligned, wherein one tab of the first member is positioned within the internal cavity of the second member and one tab of the second member is positioned within the internal cavity of the first member.

At least one flexible element 307 connects the inner surface of the tab member 305 of the first member 303 with the inner surface of the second member 303 and at least one flexible element connects the inner surface of the tab member 305 of the second member with the inner surface of the first member 303 so that said first and second members 303 may smoothly rotate relative to one another about a central longitudinal axis. The elements 307 are joined with the inner surfaces of members 303 and tabs 305 using any method that is known in the art to join these members—including welding and the like. FIG. 13B shows an exploded view of a device.

The device is commercially available from the Riverhawk company of New Hartford, N.Y. 13413. The web site http://www.flexpivots.com describes the device in detail and the totality of the information contained within the web site is hereby incorporated by reference in its entirety. Further, prior disclosures of similar flexible pivot devices have been made in U.S. Pat. Nos. 5,620,169, 6,146,044 and 6,666,612. The disclosure of each of these patents is hereby incorporated by reference in its entirety.

As previously illustrated, pivot member 515 is housed within an end of rod 502. With the longitudinal movement of collar 512 along rod 502 (that is, movement of collar 512 in the direction of the long axis of the rod 502), the interaction of cylindrical protrusion 5032 of rod 502 and straight cut-out 5128 of collar 512 prevent rotational movement of the collar relative to the long axis of the rod 502. However, since spherical protrusion 5036 is attached to the distal aspect of the pivot member 515 and retained within helical cutout 5132 of the distal aspect of collar 512, the longitudinal movement of collar 512 necessarily produces a rotational movement of the distal aspect of the pivot member 515 relative to the proximal aspect of pivot member 515 (which is fixed relative to the rod 502). In this way, longitudinal movement of collar 512 relative to rod 502 necessarily produces the rotational movement of the first and second members of the axially aligned hollow cylindrical member 303 of pivot member 515. The flat crossed slats of pivot member 515 will oppose rotational movement of the axially aligned hollow cylindrical members and resist the longitudinal movement of the collar 512 relative to rod 502. Thus, the pivot member 515 functions as a rotational resistor to longitudinal movement of the collar and rod. Animations of the moving pivot member 515 are shown on the web site http://www.flexpivots.com and all animations, drawings and specifications of the device are hereby incorporated by reference in they're entirety. Further, pivot members of three or more axially aligned hollow cylindrical members are also illustrated on site and sold by the Riverhawk Company as “double ended pivot bearings”. While the preceding invention is illustrated using a pivot member containing only two axially aligned hollow cylindrical members (sold by the Riverhawk Company as “cantilevered (single ended) pivot bearing), it is contemplated and fully understood that one of ordinary skill in the art can alternatively employ the pivot members of three or more segments to accomplish the disclosed invention. That is, any of the pivot bearings may be used to resist the longitudinal movement of collar 512 relative to rod 502 when longitudinal movement of collar 512 relative to 502 is configured to produce forcible rotation of at least one pivot bearing member relative to another pivot bearing member.

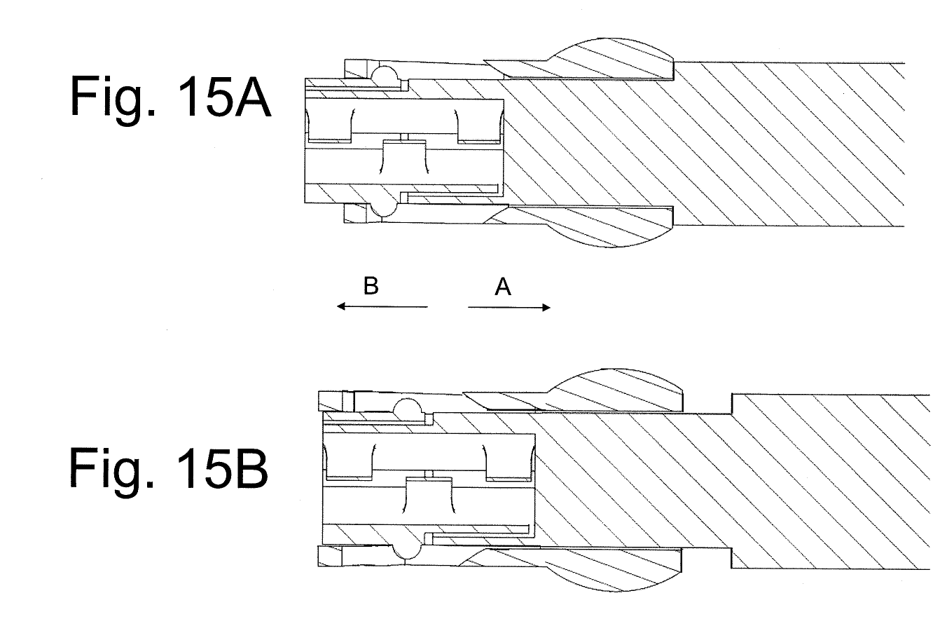

With reference to identical sectional views of FIG. 8B and FIG. 15A, collar 512 is configured to rest against the step-off between segment 5022 and segment 5024 of rod 502. In this configuration, the collar is in a “neutral” or “default” position. Movement of collar 512 (relative to rod 502) towards direction A is prevented by the step-off generated by the change from segment 5022 to segment 5024 of rod 502. Movement of collar 512 towards direction B is resisted by the pivot member 515 as previously described. In one potential use, the device is attached to a functional spinal unit (FSU) and functions to prevent vertebral extension and resist vertebral flexion—as will be illustrated below.

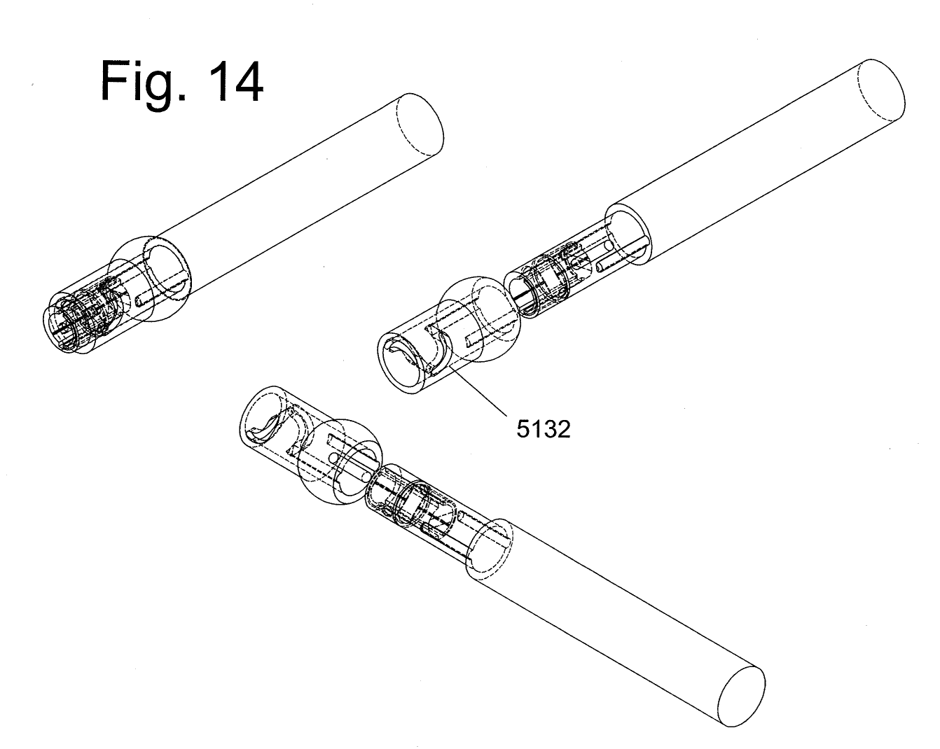

The extent of resistance to movement of collar 512 relative to rod 502 may be varied in multiple ways. In a first embodiment, resistance may be varied by modifying the flexible elements 307 of the pivot member 515, wherein the size, configuration, number, orientation, thickness or material of manufacture of the elements 307 may be changed. By way of example, shape memory alloys and/or less malleable materials (such as, for example, titanium) may be used to manufacture element 307 and will endow the device with widely varied resistance properties. Further, for any given pivot member 515, the resistance profile may be changed by altering the frictional contact between collar 512 and rod 502 or changing the shape of full thickness channel 5132 of collar 512. For example, FIGS. 13B and 14 illustrate use of curvilinear channel 5132. In this embodiment, the resistance will vary significantly and in a non-linear fashion with movement of collar 512 relative rod 502. Thus, the current invention provides exceptional control and nearly limitless variation of the resistance properties of collar movement.

FIGS. 8B and 15A show a sectional view of the first device embodiment. As noted, the device prevents movement of collar 512 (relative to rod 502) towards direction A and resists movement of collar 512 towards direction B. An alternative embodiment is illustrated in FIG. 15B, wherein movement of collar 512 (relative to rod 502) is resisted in either direction A or B by the action of pivot member 515. In this embodiment, the “neutral” position is changed to that illustrated in FIG. 15B. In the neutral or default position, protrusion 5026 is now positioned within the central aspect of channel 5132. Movement of collar 512 (relative to rod 502) towards direction A is resisted by rotation of the distal member of pivot member 515 (relative to the rod 5022) in one direction (for example, clockwise). Similarly, movement of collar 512 (relative to rod 502) towards direction B is resisted by rotation of the distal member of pivot member 515 (relative to the rod 5022) in the opposite direction (for example, counter clockwise). As discussed, the channel 5132 may be made curvilinear to change the resistance property of the device. While not shown, it is contemplated that the channel 5132 configuration may be different on each side of protrusion 5026 (when in the neutral position) so the resistance to movement towards direction A may be different from the resistance to movement towards direction B.

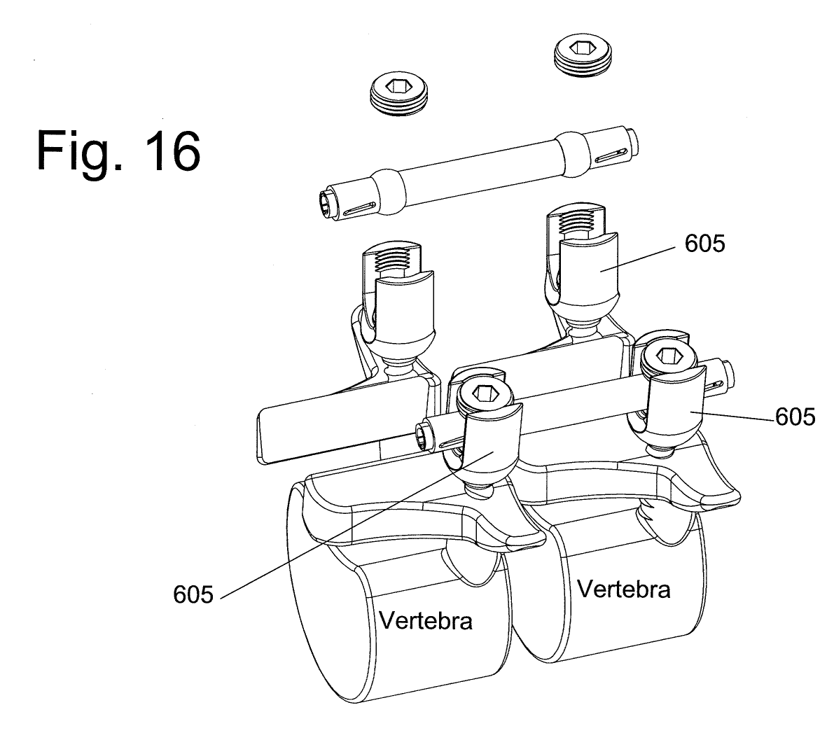

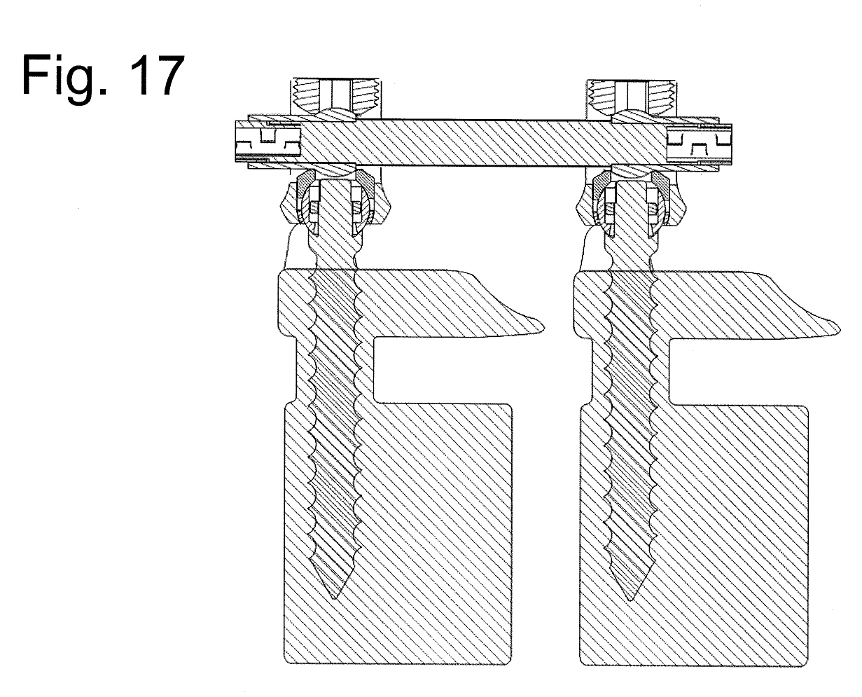

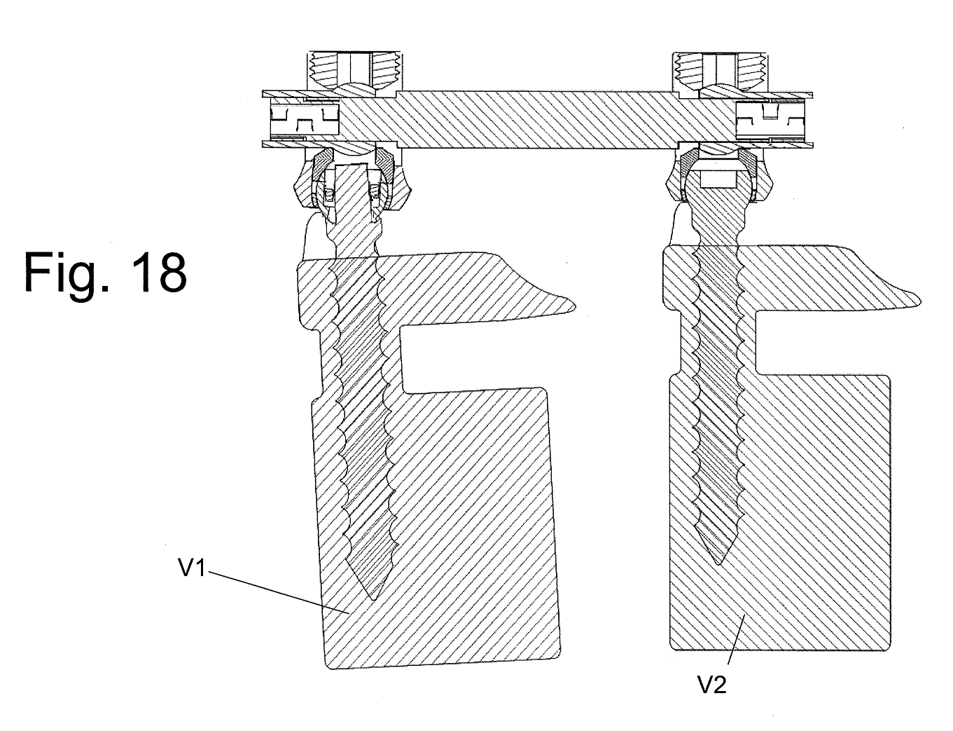

FIG. 16 illustrates a prospective view of the device attached to vertebral bone while FIG. 17 shows a sectional view through the device. (The vertebral bones are represented schematically and those skilled in the art will appreciate that actual vertebral bodies may include anatomical details that are not shown in these figures.) Note that the device is attached to bone fasteners at the spherical protrusion 5121 of the collar member 512. As such, the pivot member 515 is external to the segments of the device that interconnect the bone fasteners 605. In this way, the device retains significant strength while the resistor (i.e., pivot member 515) remains capable of resisting vertebral flexion (direction B of FIG. 15A and stopping vertebral extension (direction A of FIG. 16A). Alternatively, the device of FIG. 15B may be used to resist movement in both vertebral flexion and/or extension. FIG. 18 illustrates vertebral flexion with a rigid bone fastener used to attach the device to vertebra V2 and a movable bone fastener used to attach the device to vertebra V1.

The disclosed devices or any of their components can be made of any biologically adaptable or compatible materials. Materials considered acceptable for biological implantation are well known and include, but are not limited to, stainless steel, titanium, tantalum, combination metallic alloys, various plastics, resins, ceramics, biologically absorbable materials and the like. Any components may be also coated/made with nanotube materials to further impart unique mechanical or biological properties. In addition, any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, any surface may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. Lastly, any disclosed devices or any of its components can also be entirely or partially made of a shape memory material or other deformable/malleable material.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. An orthopedic implant adapted to resist anterior movement between a first vertebral bone and a second vertebral bone in a horizontal plane, comprising:
a first member that is adapted to affix onto the first bone;
a second member that is adapted to affix to a second bone; wherein the first and second members are adapted to move relative to each other in a longitudinal plane;
at least one flexible rotational articulation that is contained within the implant and that functions to resist at least a portion of the movement between the first and second members, the articulation having:
A first hollow cylindrical member comprised of an outer surface with a defined radius from a longitudinal central axis, an inner surface with a defined radius from a longitudinal central axis, a defined thickness and defined internal cavity that is contained within the inner surface, at least one cylindrical tab that extends from one end of said member wherein the tab circumferentially extends less than one hundred and eighty degrees around its longitudinal central axis;
a second hollow cylindrical member comprised of an outer surface with a defined radius from a longitudinal central axis, an inner surface with a defined radius from a longitudinal central axis, a defined thickness and defined internal cavity that is contained within the inner surface, at least one cylindrical tab that extends from one end of said member wherein the tab circumferentially extends less than one hundred and eighty degrees around its longitudinal central axis;
wherein the first and second members are axially aligned and wherein one tab of the first member is positioned within the internal cavity of the second member and one tab of the second member is positioned within the internal cavity of the first member;
wherein at least one flexible element connects the inner surface of the tab member of the first member with the inner surface of the second member and at least one flexible element connects the inner surface of the tab member of the second member with the inner surface of the first member so that said first and second may smoothly rotate relative to one another about a central longitudinal axis.
